(19) United States
(12) Patent Application Publication
(10) Pub. No.: US 2009/0162405 A1
(43) Pub. Date: Jun. 25, 2009

(54) Proteinase-engineered cancer vaccine induces immune responses to prevent cancer and to systemically kill cancer cells

(76) Inventor: Yong Qian, San Diego, CA (US)

Correspondence Address: Yong Qian, Biomedicure LLC, 7933 Silverton Ave, Suite 711, San Diego, CA, 92126, US

(21) Appl. No.: 12/322,237

(22) Filed: Jan. 31, 2009

Related U.S. Application Data
(63) Continuation-in-part of application No. 11/638,747, filed on Dec. 14, 2006, now abandoned.

(51) Int. Cl. A61K 39/00

(52) U.S. Cl. 424/277.1

(57) ABSTRACT

A harmless cancer vaccine is made from cancer cells with extracellular proteins including self-recognition molecular patterns being digested by a proteinase. The cancer vaccine is used to vaccinate an individual to induce immune responses against cancer cells systemically. Cancer cells become harmless when they are digested by Tumorase™. Some proteinases including trypsin cannot kill cancer cells completely and treated cancer cells need to be further processed in order to be harmless and effective. Cancer cells may be from tissue-cultured human or animal cancer cell lines or cancer patients directly. Cancer vaccine vaccinated individuals produce cancer vaccine specific immune responses against cancer cells. Immune response components may be isolated and used to fight against cancer for a cancer patient with a suppressed immune system. Cancer vaccine specific immune components may include cancer vaccine specific polyclonal antibodies, B-cells, T-cells, natural killer cells, monocytes, macrophages and other lymphocytes.

### CROSS-REFERENCE TO RELATED APPLICATIONS

1) This utility patent application in part is the continuation of application Ser. No. 11/638,747 titled “Bioknives-aided cytoreductive immunotherapy system for solid-tumors filed on Dec. 14, 2006 by Yong Qian, Biomedicure LLC.

2) This patent application claims the benefit of patent application Ser. No. 11/825,246 titled “Proteinases destroy cancer tumor's solid structure and kill cancer cells locally” filed on Jul. 5, 2007 by Yong Qian, Biomedicure LLC. However, this patent is not the continuation of application Ser. No. 11/825,246.

### STATEMENT REGARDING FEDERALLY SPONSORED RESEARCH OR DEVELOPMENT

This invention was not sponsored by any federal research or development fund.

### REFERENCE TO SEQUENCE LISTING, A TABLE, OR A COMPUTER PROGRAM LISTING COMPACT DISC APPENDIX

Not Applicable

### BACKGROUND OF THE INVENTION

The idea of using proteinases to do solid-tumor microsurgery has led to the discovery of a new class of drugs that can eliminate solid-tumors by destroying the solid-structure of the main tissue of the tumor and kill actively-dividing cells locally⁽²⁾. Basically, proteinases are employed to digest extracellular proteins including the extracellular domains of cell membrane proteins within a tumor. This kills actively dividing cells including cancer cells locally so as to eliminate a tumor as an organ. Desired outcomes are to eliminate tumor organs before cancer metastasis. However, due to some known reasons (such as irregular tumor shapes, locations, types and stages of the cancer, micrometastasis, proteinase species used and the surrounding tissue or organ microenvironment around the tumor organ) and other unknown reasons, the proteinase biochemotherapy may not be able to kill all cancer cells, especially in cases of deep tumors, malignant soft tumors and micrometastasis. The untreated cancer cells may continue to grow and to metastasize to form new tumor organs. If the immune system is programmed with information against cancer cells by previous vaccination with a cancer vaccine or cancer vaccines, the proteinase biochemotherapy would be more effective because the immune system will kill any untreated or metastasized cancer cells for potential cure.

Cancer causes, types, races, diagnoses, treatments and challenges have been previously described^{(1,2)}. However, challenges in developing an immunotherapy to treat cancer patients can be further addressed. First of all, a solid-tumor is an organ composed of a main tissue of cancer cells packed and networked together by over-expressed extracellular proteins which form a solid structure, and sporadic tissues of actively-dividing normal cells and blood vessels. Sporadic tissues were recruited by the main tissue to support the growth of the tumor organ. Secondly, the solid-structure of the main tissue of the tumor organ traps macrophages to disrupt their antigen-presentation processes. Thirdly, the tumor organ expresses and over-expresses cytokines and interleukins that drive immune screening cells including dendritic cells, B-cells, T-cells, natural killer cells and monocytes away from the organ. These events further disrupt the immune system's antigen sampling and presentation processes. Fourthly, the expression and over-expression of self-recognition molecular patterns by cancer cells prevents the immune system from obtaining cancer cells' mutation information. Thus, chemotherapy small molecules, immunotherapy monoclonal antibodies and T-cells are not effective enough against cancer if the tumor organ is not disrupted or eliminated. Proteinase-based biochemotherapy can quickly (within hours) and effectively eliminate the malignant solid-tumor organ locally⁽²⁾. However, the immune system takes weeks to work pro-actively against cancer cells. There is an urgent need to pre-program the immune system to fight against cancer cells more quickly. Furthermore, the difference between extracellular matrices of cancer cells and that of actively dividing normal cells is not significant enough for the immune system to recognize. There is a great need to alter the self-recognition molecular patterns on the surfaces of cancer cells and expose their cancer cell specific mutation information for the body's immune system (via various lymphocytes) to recognize, sample, present, compare, process and eventually memorize in order to make cancer vaccine induced immune responses working against cancer cells.

### BRIEF SUMMARY OF THE INVENTION

A proteinase-engineered harmless cancer vaccine is invented for prevention and potential cure of cancer. A proteinase is used to make a cancer vaccine by altering cancer cells' self-recognition molecular patterns on cancer cell surfaces leaving the cell membrane intact. The vaccine is harmless to normal healthy cells and will not transform normal cells to cancer cells. The cancer vaccine induces immune responses against cancer cells using shared mutation information in the vaccine and cancer cells. The cancer vaccine may be used for cancer prevention for both healthy and pre-cancer high-risk individuals. It can be used as an immunotherapy drug for a cancer patient if the genetic or antigen mutation information in the cancer vaccine is the same or similar to that in the patient's cancer cells. The vaccine may also be useful for cancer patients who may undergo biochemotherapy using the same or different proteinase agent(s) for solid-tumor elimination locally because proteinases can disrupt or destroy the solid-structure of a malignant solid tumor and the cancer vaccine induced immune responses can kill any remaining cancer cells for a potential cure. Furthermore, some proteinases can kill cancer cells directly and others cannot⁽²⁾, those that are not able to kill cancer cells by themselves may be used to destroy the solid-structure of malignant solid tumor organs in immunized cancer patients allowing the immune system to kill remaining cancer cells for a potential cure. The proteinase agent may be any proteinase that can alter the conservative self-recognition molecular patterns of cancer cells but maintain mutation information in their cancer associated antigens which may include but is not limited to expression of one to multiple onco-genes, loss of tumor suppressor genes, tumor promoting microRNAs, heterogeneous, unstable or mutating genomes and associated gene over-expression patterns.

Cancer vaccines may be made from cancer cells that derived from tissue-cultures or from cancer patients directly. When these vaccines are used to immunize healthy or high-risk individuals, cancer cell mutation information is entered into their immune systems. These systems will be able to kill cancer cells according to their acquired mutation information. Thus, cancer within the mutation range of the cancer vaccine will be prevented. The cancer vaccine specific immune components including polyclonal antibodies made against cancer vaccines, and lymphocytes including B-cells, natural killer cells, T-cells and macrophages involved in the immune responses against target cancer cells, may be obtained from the blood of immunized individuals. Concentrated or purified cancer vaccine specific immune components may be used as therapeutic agents to help a cancer patient's immune system to fight against cancer cells. Individual animal or human cancer patients may be injected with the cancer vaccine via subcutaneous (sub-Q) once a week for five consecutive weeks or more until all cancer cells are killed. When needed, multiple cancer vaccines may be used to vaccinate cancer patients and healthy individuals as well. A local biochemotherapy tumor elimination drug such as Tumorase™ or other proteinase agents may be used in combination with the cancer vaccine to eliminate malignant solid tumor organs. When most of, if not all, malignant solid-tumor cancer cells are digested extracellularly by a proteinase, they will be killed either by the proteinase agent or the activated immune responses. These and other objects, advantages, and features of the invention will be better understood by reference to the several views of drawings and the detailed descriptions of the invention.

### BRIEF DESCRIPTION OF THE SEVERAL VIEWS OF THE DRAWING

For a better understanding of the present invention, and to show more clearly how the same may be carried into effect, reference will be made to the accompanying drawings.

FIG. 1 is a schematic illustration of using a proteinase agent to create a harmless cancer vaccine capable of inducing immune responses against cancer cells.

FIG. 2 is a schematic illustration of using the cancer vaccine for cancer prevention in healthy or high-risk pre-cancer individuals and the use of the vaccine or the cancer vaccine specific immune components to kill cancer cells.

FIG. 3 is a tumor growth chart showing cancer vaccine vaccinated male mice induced immune responses against malignant tumor cancer cells vs. unvaccinated male mice which did not induce immune responses against cancer cells' malignant tumor growth.

FIG. 4 is a tumor growth chart showing cancer vaccine vaccinated female mice induced immune responses against cancer cells' malignant tumor growth vs. unvaccinated female mice which did not induce immune responses against cancer cells' malignant tumor growth.

FIG. 5 is a tumor growth chart showing cancer vaccine vaccinated mice induced immune responses against cancer cells' malignant tumor growth vs. normal cell “vaccine” vaccinated mice and unvaccinated mice which did not induce immune responses against cancer cells' malignant tumor growth.

### DETAILED DESCRIPTION OF THE INVENTION

Vaccine refers to a harmless variant or derivative of a pathogen that is presented to the body in order to induce an immune response against the pathogen. A cancer vaccine refers to harmless variants or derivatives of cancer cells that are presented to the body in order to induce immune responses against cancer cells for cancer prevention or immunotherapy of active cancers. The cancer vaccine is composed of variants or derivatives of cancer cells because cancer cells are heterogeneous and mutating cells that are not a clone of the same cells or a mixture of several cancer clones. Thus, a cancer vaccine induces immune responses (not a single immune response) against cancer cells. Furthermore, a singer cancer vaccine may induce limited immune responses depending on the mutation information contained in the vaccine. Multiple cancer vaccines may be used for multiple cancer prevention or treatments. Targeted cancers may include any forms including cancer cells not forming tumors, cancer cells in malignant tumors, micrometastasis, matastasis and cancer neoplasm located in different organs of the body. Cancer vaccine specific immune responses may be studied in laboratory and clinical trials. Research and development results can be further applied to in vitro, in vivo and clinical studies using cancer cell cultures (suspension or attached), tissue cultures, organ cultures, nude and wild-type mice models and clinical trials.

The cancer mutation information is built into the cancer cells' heterogeneous and unstable genomes and expressed in their gene expression patterns including but not limited to one to tens of onco-gene expressions, loss of the tumor-suppressor gene expressions, production of microRNAs that promote tumor formation and expression of tumor-associated antigens and immune suppressing genes. Therefore, one cancer vaccine may induce immune responses to kill the majority of cancer cells from which the cancer vaccine is derived from, but the immune responses may not be able to kill all cancer cells if cancer cells mutate further beyond the information contained in the cancer vaccine.

Cancer vaccine is still a concept because there is no successful example yet. Gardasil and Cervarix are vaccines used to prevent cancer such as cervical cancer caused by the human papillomavirus (HPV). These vaccines are not cancer vaccines because they are not derivatives of any cancer cells and cannot be used to induce any immune responses against cancer cells including cervical cancer cells. When they are presented to the body, Gardasil and Cervarix induce an immune response against the HPV virus and to prevent the HPV viral infection and associated diseases including cervical cancer. Thus, to qualify as a cancer vaccine, first it has to be variants or derivatives of cancer cells or tumor organs. Secondly, it has to be harmless to normal or healthy cells or the body and does not transform any normal cells to cancer cells. Thirdly, it must have the capability to induce immune responses against cancer cells.

So far, there is no successful example although many “cancer vaccines” have already advanced to late stage clinical trials. One possible reason for the failure of “cancer vaccines” is that the tested “cancer vaccines” might not induce immune responses because their self-recognition molecular patterns prevent them from being recognized by, or presented to, the immune system. Other possible reasons may be one or the combination of the following: 1) cancer cells were killed by y-ray to make “cancer vaccines” harmless. However, the y-ray fragmented DNA (into small pieces) may never match the genetic mutation information in target cancer cells. The “cancer vaccines” may thus confuse the immune system. 2) y-rays may also cause protein cross-links that do not match antigens on the cell surface, in cell membrane or inside target cancer cells. 3) the self-recognition molecular patterns on the cell surface of “cancer vaccines” are different from normal cells of test animal models and induce strong immune responses in animal models but not in human beings. If “cancer vaccines” were effective, other factors including the over-expression of the self-recognition molecular patterns, cytokines and interleukins by malignant solid tumor organs may still prevent or suppress the immune responses.

A malignant tumor organ with a solid-structured main tissue and sporadic tissues might be more complicated than what we currently understand scientifically, physiologically and systemically. Indeed, many mechanisms at the body system level are different from mechanisms at the organ, tissue, cell and molecular levels due to compartmentation, blood flow direction and cycling, and interactions among different organs. The mutating and heterogenic nature of cancer cells may be the root of the problem. This information has to be entered and remembered by the immune system in order for the system to work against cancer cells for prevention and potential cure of cancer.

When digested by Tumorase™, tissue-cultured cancer cells were found dead and harmless. 120 nude mice (60 males and 60 females) did not grow any tumor after they were injected with 4×10⁶ Tumorase™-treated cancer cells with intact cell membranes⁽²⁾. It was not known if they could induce immune responses against cancer cells because nude mice did not have intact immune systems. Thus, wild-type mice are used to test if Tumorase™-treated cancer cell derivatives can induce immune responses against genetically compatible wild-type mice cancer cells from which the cancer vaccine was derived.

Because self-recognition molecular patterns including major histocompability complex (MHC) are extracellular proteins, a proteinase that digests self-recognition molecular patterns can be used to digest tissue-cultured cancer cells' extracellular matrix proteins and to make cancer vaccines conveniently. The proteinase may also be used to digest cancer cells or tumors from a cancer patient directly to make a personal cancer vaccine that may trigger immune responses to prevent recurrence of the same cancer.

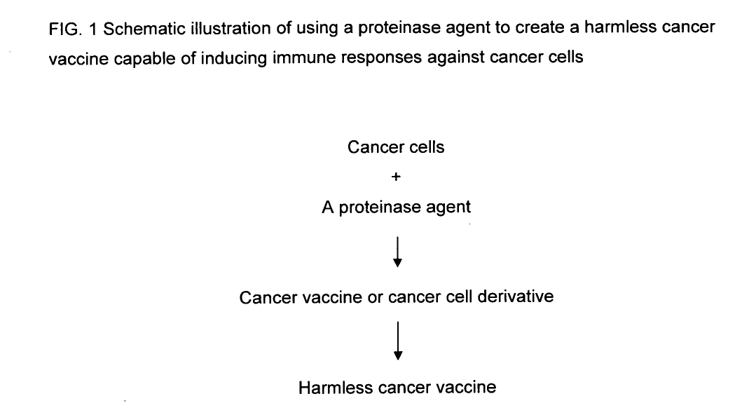

FIG. 1 is a schematic illustration of using a proteinase agent to create a harmless cancer vaccine capable of inducing immune responses against cancer cells. Cancer cells may be from tissue cultures or tumors of a cancer patient directly. If they are from tissue cultures, cancer cells are grown in flasks with appropriate medium, serum, pH, temperature, CO₂ concentration and humidity for optimal growth. When cancer cells are crowded, the medium is decanted and washed them with a buffer or a small amount of a proteinase solution to eliminate proteinase inhibitors and to generate an optimal condition for the action of the proteinase agent. The proteinase agent cleaves peptide bonds on extracellular matrix proteins C-terminally, N-terminally or both depending on the species and the number of proteinases used. Cancer cells are separated individually and released from the container walls or adjacent cells as well. These cancer cells are briefly centrifuged to pellet and the supernatant is decanted. The pellet is re-suspended and washed two more times with phosphate buffer saline (PBS) and repeated centrifugation to eliminate amino acids, peptides and the proteinase agent completely. If cancer cell derivatives are dead as seen with the Tumorase™ treatment, they can be used as a cancer vaccine directly. If the cells are still alive as seen with the trypsin treatments, cancer cell derivatives can be further processed to make the cancer vaccine harmless by treating with formalin, phenol, a combination of freeze-thaw, heat and freeze, or other means with proper storage. If cancer cells are from tumors of a cancer patient directly, a biosurgery or a biochemotherpy^{(1,2)} may be used to obtain cancer cells. A large tumor or multiple tumors from a conventional surgery of a cancer patient may also be treated with a proteinase such as Tumorase™ to make a harmless cancer vaccine. The cancer patient may be human or any animal under medical care. Cancer cells may also come from other sources including but not limited to cancer cell suspension culture, cancer tissue or organ culture in vitro or in vivo in nude mouse models or other animals that are immune deficient.

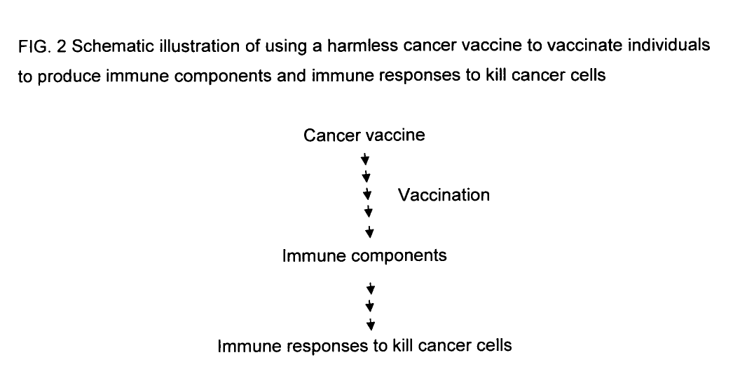

FIG. 2 is a schematic illustration of the use of cancer vaccine and the cancer specific immune components to prevent cancer and to kill existing cancer cells. A cancer vaccine can be directly used to vaccinate healthy individuals or pre-cancer high risk individuals to induce the production of immune components ready for immune responses against cancer cells. The cancer vaccine specific immune components may be isolated from the vaccinated individuals via their blood draw or donation. Concentrated or purified cancer vaccine specific immune components including polyclonal antibodies, B-cells, macrophages, T-cells and other lymphocytes may be injected to a cancer patient's blood directly for immunotherapy against cancer cells. Vaccinated individuals may be human or animals including, but not limited to, mouse, dog, cat, hamster, horse, rabbit, rat, chicken, cow, tiger, panda, pig, sheep and monkey. The same cancer vaccine may be studied in different species. All species, including the species where the cancer vaccine is come from, should have and will have immune responses. However, the cancer vaccine specific immune components involved in the induced immune responses are different. For example, lymphocytes including T-cells, natural killer cells, monocytes, dendritic cells, macrophages and B-cells from mice are different from those in human. However, some of the polyclonal antibodies against the cancer vaccine are specific and may be common. It is valuable to find the common polyclonal antibodies so as to make them in animals and to isolate them for human use.

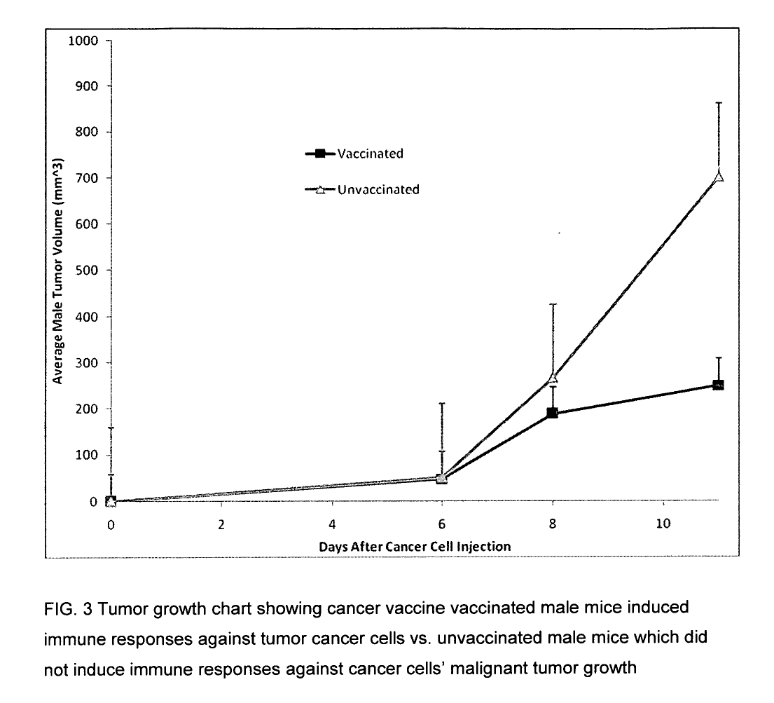

FIG. 3 is a tumor growth chart showing cancer vaccine vaccinated male mice induced immune responses against malignant tumor cancer cells vs. unvaccinated male mice which did not induce immune responses against cancer cells' malignant tumor growth. Reduced tumor growth volume in vaccinated mice was the direct result of cancer vaccine induced immune responses against injected cancer cells. Cancer vaccine specific immune components including polyclonal antibodies and lymphocytes such as B-cells, T-cells, natural killer cells, monocytes and macrophages are major contributors of the immune responses against cancer cells injected. The cancer vaccine specific immune responses limited the cancer cell composition and population of the tumor main tissue and restricted their recruiting activity of normal cells in the sporadic tissues. The structure of the tumor changed from the irregular to confined or solid forms. This further reduces the micrometastasis of cancer cells and increases the life quality and span of the mice. Thus, cancer vaccines solve the problems Tumorase™ based biochemotherary has in treating systemic micrometastasis, metastasis, deep tumors and irregular shaped soft malignant tumors.

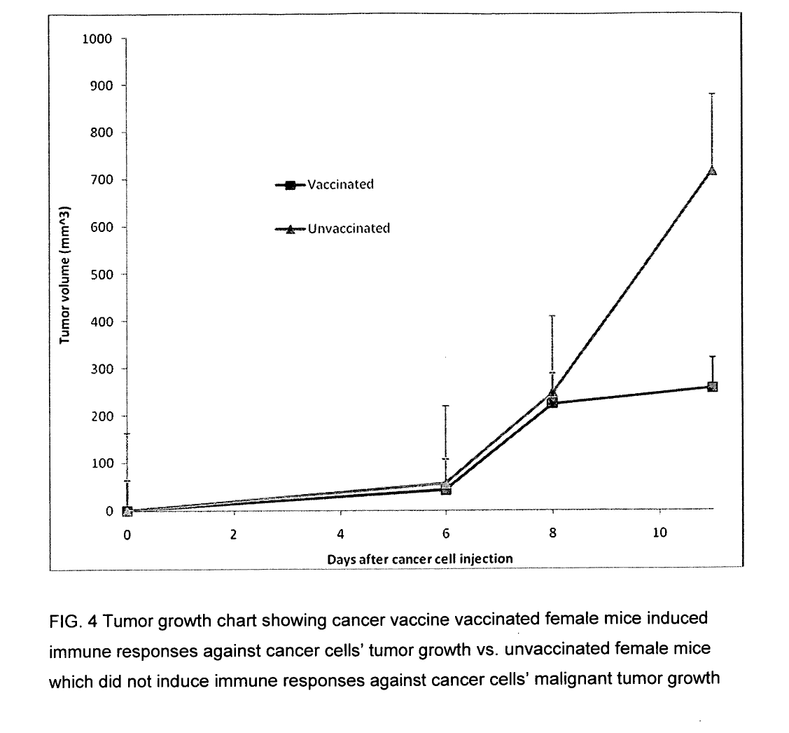

FIG. 4 is a tumor growth chart showing vaccinated female mice induced immune responses against malignant tumor cancer cells vs. unvaccinated female mice which did not induce immune responses against cancer cells' malignant tumor growth. In a combination with other therapies including Tumorase™ biochemotherapy and conventional surgery to remove solid-tumor organs, cancer vaccine immunotherapy will play very important role for cancer treatment. When multiple cancer vaccines against most cancer types are used for vaccination, cancer may be prevented, treated as a group of curable immune deficient diseases.

Detailed experimental procedures for cancer cell culture, cancer vaccine small-scale production, cancer vaccine vaccination, cancer cell injection, and tumor measurement are as follows.

A mouse melanoma tumor cell line (CRL-6475, ATCC, Manassas, Va.) has been cultured in flasks containing 60 ml Eagle's Minimum Essential Medium (30-2003, ATCC, Manassas, Va.) with 5% fetal bovine serum USDA Premium (9871-5200, USA Scientific, Ocala, Fla.) under conditions previously described⁽²⁾. Crowded cancer cells were separated by 0.25% 1× Trypsin (Invitrogen, Carlsbad, Calif.) and subcultured. Tumorase™ (Biomedicure, San Diego, Calif.) was used to harvest the subcultured cancer cells to make a cancer vaccine in PBS after three times PBS washes by centrifugation for 10 minutes each at 1000 revolutions per minute (RPM) using a clinical centrifuge. The cancer vaccine contains about 2×10⁷ dead cancer cells per 1 ml. It can be used immediately or stored at −20° C. for future use.

Wild-type mice (B16-F10, 23 days old) were purchased from Charles River (Hollister, Calif.) and delivered to the ovarian facility at Bio-Quant, Inc (San Diego, Calif.). Five male mice (31 days old) and five female mice (31 days old) were sub-Q injected with the cancer vaccine (about 2 million dead cancer cells) in 100 uL PBS three times when the mice were 31, 38 and 45 days old. Other 5 male and 5 female mice (the same age) did not receive any cancer vaccine injection and served as control groups.

The same melanoma tumor cell line (as was used to make cancer vaccine) was harvested with the same trypsin solution above and used to grow tumors in both vaccinated and unvaccinated mice (20) randomly. About 1×10⁶ cancer cells were injected via sub-Q on each of two sites of the flank of a randomly selected mouse when they were 54 days old.

Tumors were two dimensionally measured using an electronic caliper on days 6, 8 and 11 after cancer cell injections. Tumor volume was calculated by ½ ab² in mm³ volume where “a” represents the tumor length in mm and “b” is the tumor width in mm measured.

In FIG. 3, the unvaccinated male control group had tumors grew faster 8 days after the cancer cell injection than tumors on the cancer vaccine vaccinated male group. The average tumor volume for the unvaccinated male control group was about 702 mm³ 11 days after the cancer cell injection while the average tumor volume for the cancer vaccine vaccinated male group was about 250 mm³ 11 days past the cancer cell injection.

The unvaccinated female control group had tumors grew faster 8 days after the cancer cell injection than tumors on the cancer vaccine vaccinated female group. The average tumor volume for the unvaccinated female control group was about 715 mm³ 11 days after the cancer cell injection while the average tumor volume for the cancer vaccine vaccinated female group was about 264 mm³ 11 days past the cancer cell injection.

Thus, the average tumor volume for the unvaccinated control groups (5 males and five females) were about 708 mm³ 11 days past the cancer cell injection while the average tumor volume for the cancer vaccine vaccinated groups (5 male and 5 females) were about 257 mm³ 11 days past the cancer cell injection (FIG. 4).

The cancer vaccine vaccination have induced vaccinated animals' immune responses against cancer cells (1 million per site, 2 million per animal) injected by sub-Q. Because there was no tumor grown on any vaccinated mice before cancer cell injection and there were no significant weight changes for any vaccinated animals when compared with unvaccinated animals (data not shown), the cancer vaccine did not show any adverse effects.

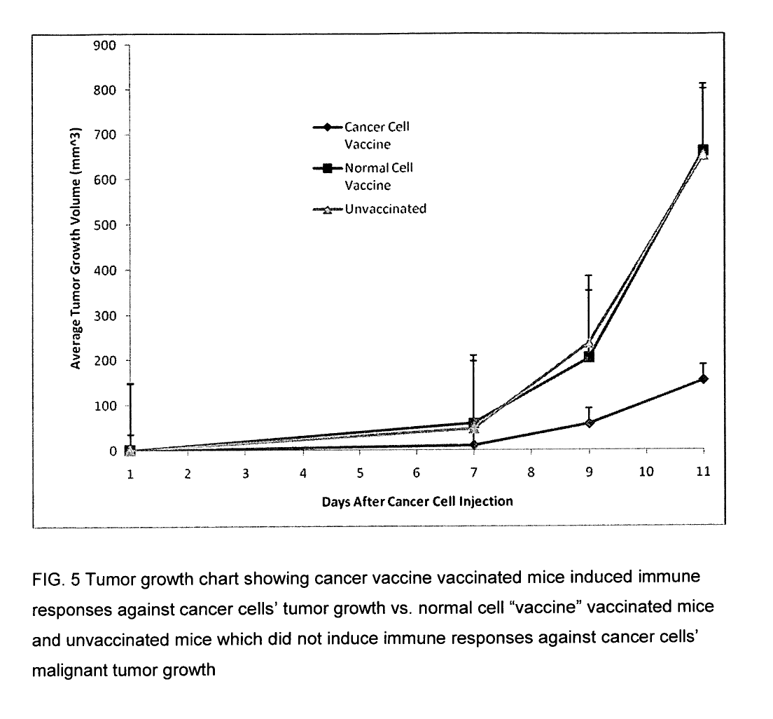

FIG. 5 further showed that cancer vaccine vaccinated male and female mice have induced immune responses against cancer cells' malignant tumor growth while normal cell “vaccine” vaccinated mice and unvaccinated mice did not induce immune responses against cancer cells' malignant tumor growth. The normal cell “vaccine” was made by the same procedure used to make cancer vaccine except using tissue-cultured cells from a normal mouse epidermis cell line (CRL-2007, ATCC, Manassas, Va.). Details of experiment procedures are similar to those of the previous experiment.

Nine mice (4 males, 5 females, 65 days old) were sub-Q injected with the same cancer vaccine (about 1.75 million dead cancer cells per mice) in 100 μL PBS 5 times when the mice were 65, 72, 79, 86 and 91 days old.

Nine mice (4 males, 5 females, 65 days old) were sub-Q injected with the normal cell derived “vaccine” (about 2.6 million dead cells per mice) in 100 uL PBS 5 times when the mice were 65, 72, 79, 86 and 91 days old.

Nine mice (4 males, 5 females, 65 days old) were sub-Q injected with 100 uL PBS 5 times when the mice were 65, 72, 79, 86 and 91 days old.

The same melanoma cancer cell line described in the previous experiment was prepared and used to sub-Q inject each of the 27 mice randomly selected when they were 105 days old. Every mouse had about 1×10⁶ cancer cells injected in 100 uL PBS suspensions.

Tumors were two dimensionally measured with an electronic caliper on days 7, 9 and 11 after cancer cell injections. Tumor volume was calculated the same way as described above.

In FIG. 5, the normal cell derived “vaccine” vaccinated mice showed similar tumor growth curve to that of the control without any vaccination. On day 11 after the cancer cell injection, the cancer vaccine vaccinated group showed significantly lower average tumor volume (about 155 mm³) than that of control (about 653 mm³) and that of normal cell “vaccine” control (about 663 mm³). However, the average tumor volume between the unvaccinated and the normal cell “vaccine” vaccinated animal groups were not significantly different at any point recorded.

When comparing results from the first experiment (FIG. 4) and the second experiment (FIG. 5), the average tumor volume for control groups at different experiments was similar. However, the cancer vaccine vaccinated group with 5 vaccinations in 5 consecutive-weeks (FIG. 5) showed better immune responses than the group vaccinated 3 times in 3 consecutive-weeks (FIG. 4). This is reasonable because the longer the cancer vaccine presented to the mice, the more mutation information in the cancer vaccine may be entered into mice's immune system and stronger immune responses have been shown. Vaccinated animals not only have smaller tumors but also have movable tumors which be easily eliminated by Tumorase™ biochemotherapy or conventional operations. Furthermore, multiple cancer vaccines' vaccinations may enable the vaccinated acquire total immunity against all cancer cells in the tumor.

The detailed mechanism of immune responses induced by the cancer vaccine is unknown. However, several things are sure. First of all, the cancer vaccine is foreign to the immune system because their cell surfaces do not have the self-recognition molecular patterns (cleaved off by Tumorase™ during preparation). This enabled lymphocytes including dendritic cells and macrophages to recognize them, sample them and present their antigen profile to the immune system. Secondly, the mutation information in the cancer vaccine might be presented to T-cells through antigen-presentation processes by dendritic cells and macrophages. Thirdly, the mutation information within the antigen profile was compared to those in normal cells, retained and memorized by B-cells. Fourthly, polyclonal antibodies against cancer vaccine specific antigens might be produced. In the presence of living cancer cells, polyclonal antibodies may bind to cancer cells to induce antibody-dependent cellular cytotoxicity (ADCC). Furthermore, the presence of cancer cells may also trigger the proliferation of lymphocytes including B-cells, T-cells and natural killer cells and more polyclonal antibodies production to immune against cancer cells.

In addition to Tumorase™, other proteinases including carboxypeptidase B, elastase, plasmin, endoproteinase Glu-C, endoproteinase Asp-N, endoproteinase Lys-C, endoproteinase Arg-C, chymotrypsin, or carboxypeptidase Y, caspases, proteinase K, subtilisin BL, M-protease, thermitase, subtilisin Carlsberg, subtilisin Novo BPN′, subtilisin BPN′, selenosubtilisin, tonin, blood coagulation factor XA, rat mast cell protease II, kallikrein A, pronase, trypsin, anhydro-trypsin, beta-trypsin, alpha-chymotrypsin, gamma-chymotrypsin, elastase, tosyl-elastase, human neutrophil elastase, human leukocyte elastase, alpha-thrombin, gamma-thrombin, epsilon-thrombin, glutamic acid specific protease, achromobacter protease I, alpha-lytic protease, proteinase A, proteinase B, actinidin, cathepsin B, papaya protease omega, papain, interleukin 1-beta converting enzyme, myeloblastosis associated viral protease, rous sarcoma virus protease, simian immunodeficiency virus protease, HIV-1 protease, HIV-2 protease, cathepsin D, chymosin B, endothiapepsin, penicillopepsin, pepsin, pepsin 3A, renin, rhizopuspepsin, neutral protease, thermolysin, astacin, astacin (zinc replaced by Cu2+), astacin (zinc replaced by cobalt2+), astacin (zinc replaced by mercury2+), astacin (zinc removed), astacin (zinc replaced by nickel2+), serralysin (bound to zinc), collagenase, fibroblast collagenase and neutrophil collagenase might also be used to make cancer vaccines out of cancer cells because they can change the self-recognition molecular patterns on cancer cell surfaces as well.

Because these proteinases will digest cancer cell surface proteins to various degrees, some cancer cells may not be killed by their digestions. Other methods including formalin, phenol, heat, freeze-thaw-freeze, y-ray, x-ray, microwave and UV may be used to make the cancer vaccine harmless. For example, when proteinase trypsin is used to digest tissue-cultured cancer cells, cancer cells may survive and continue to grow when the environment is right, although their self-recognition molecular patterns are altered. To make the trypsin digested cancer cells a cancer vaccine, the digested cancer cells need to be further processed by formalin, phenol, heat, freeze-thaw-freeze, y-ray, x-ray, microwave, UV or another proteinase digestion. Basically, trypsin digests cancer cells' extracellular matrix proteins into pieces by cutting between argenine and lysine amino acid sequences. This action is not enough to kill the cells. Thus, other proteinases that are similar to trypsin may need similar procedures to make the cancer vaccine harmless. Inject cancer vaccines to healthy individuals will eventually prove it the vaccine is harmless to the intended users.

Because a cancer vaccine can induce immune responses against cancer cells, limiting the growth of tumors but not killing all cancer cells, it is appropriate to use a proteinase biochemotherapy to disrupt or destroy the solid-structure of the tumor and systemically kill all cancer cells. Although the site-specific proteinases themselves may not be able to kill cancer cells, additional immune responses will kill living cancer cells with changes on their self-recognition molecular patterns. Thus, a combination of cancer vaccine or vaccines with less toxic proteinase's biochemotherapy on tumors has great potential to eliminate cancer cells from human or animal.

Because cancer vaccine can induce immune responses against cancer cells, the vaccine can be used to prevent cancer in healthy individuals or pre-cancer high-risk individuals. These individuals may be human or animals if cancer vaccines were made from tissue-cultures of human or animal cancer cell lines selected from the following (next 4 pages): human cancer cell lines including cervix adenocarcinoma (HeLa, ATCC), colon adenocarcinoma (TAC-1, ATCC), duodenum adenocarcinoma (HuTu 80, ATCC), endometrium uterus adenocarcinoma (KLE, ATCC), kidney adenocarcinoma (A704, ATCC), lung adenocarcinoma (NC1-H1373, ATCC), mammary gland adenocarcinoma (Hs 274.T, ATCC), ovary adenocarcinoma (Caov-3, ATCC), pancreas adenocarcinoma (BxPC-3, ATCC), rectum adenocarcinoma (SW837, ATCC), lung bronchogenic adenocarcinoma (Hs229.T, ATCC), cecum colorectal adenocarcinoma (NC1-H716, ATCC), colon colorectal adenocarcinoma (HCT-15, ATCC), rectum colorectal adenocarcinoma (SW1463, ATCC), pancreas ductal adenocarcinoma (PL45, ATCC), transfected prostate adenocarcinoma (CA-HPV-10, ATCC), stomach gastric adenocarcinoma (AGS, ATCC), non-small cell lung cancer adenocarcinoma (NC1-H23, ATCC), kidney renal adenocarcinoma (ACHN, ATCC), mammary gland scirrhous adenocarcinoma (Hs 742.T, ATCC), skin hereditary adenomatosis (182-PF SK, ATCC), kidney angiomyolipoma (SV7tert, ATCC), brain astrocytoma (CCF-STTG1, ATCC), nipple breast cancer (HT 762.T, ATCC), lung cancer (Hs 573.T, ATCC), non-small cell lung cancer (NC1-H2135, ATCC), mammary gland cancer (Hs 319.T, ATCC), colon colorectal cancer (Hs 675.T, ATCC), lung carcinoid (NC1-H835, ATCC), cortex adrenal gland carcinoma (NC1-H295R, ATCC), urinary bladder carcinoma (Hs 195.T, ATCC), cervix carcinoma (C-4 I, ATCC), kidney carcinoma (A-498, ATCC), lung carcinoma (A549, ATCC), mammary gland carcinoma (Hs 540.T, ATCC), ovary carcinoma (Hs 38.T, ATCC), pancreas carcinoma (MIA PaCa-2, ATCC), prostate carcinoma (22Rv1, ATCC), stomach carcinoma (Hs 740.T, ATCC), endometrium uterus carcinoma (RL95-2, ATCC), lung adenosquamous carcinoma (NC1-H596, ATCC), cortex adrenocortical adrenal gland carcinoma (NC1-H295, ATCC), lung alveolar cell carcinoma (SW 1573, ATCC), skin basal cell carcinoma (TE 354.T, ATCC), lung classic small cell lung cancer carcinoma (NC1-H1688, ATCC), kidney clear cell carcinoma (Caki-2, ATCC), ovary clear cell carcinoma (ES-2, ATCC), cecum colorectal carcinoma (SNU-C2B, ATCC), colon colorectal carcinoma (HCT 116, ATCC), rectum colorectal carcinoma (Hs 722.T, ATCC), mammary gland ductal carcinoma (UACC-812, ATCC), testis embryonal carcinoma (Cates-1B, ATCC), epidermoid carcinoma (A431, ATCC), lung epidermoid carcinoma (HLF-a, ATCC), duct pancreas epithelioid carcinoma (PANC-1, ATCC), stomach gastric carcinoma (SNU-1, ATCC), liver hepatocellular carcinoma (SNU-398, ATCC), medulla thyroid carcinoma (TT, ATCC), liver pleomorphic hepatocellular carcinoma (SNU-423, ATCC), mammary gland primary ductal carcinoma (HCC38, ATCC), mammary gland primary metaplastic carcinoma (HCC1569, ATCC), small cell lung cancer carcinoma (DMS 53, ATCC), cervix squamous cell carcinoma (SW756, ATCC), lung squamous cell carcinoma (SW 900, ATCC), pharynx squamous cell carcinoma (FaDu, ATCC), thyroid squamous cell carcinoma (SW579, ATCC), tongue squamous cell carcinoma (SCC-15, ATCC), vulva squamous cell carcinoma (SW 954, ATCC), urinary bladder transitional cell carcinoma (UM-UC-3, ATCC), ureter transitional cell carcinoma (Hs 789.T, ATCC), bone chondrosarcoma (Hs 819.T, ATCC), placenta chondrosarcoma (JAR, ATCC), skin dermatofibrosarcoma (Hs 357.T, ATCC), skin dermatofibrosarcoma protuberans (Hs 295.T, ATCC), erythroblast bone marrow erythroleukemia (TF-1, ATCC), connective tissue fibrosarcoma (HT-1080, ATCC), brain glioblastoma (A172, ATCC), brain astrocytoma glioblastoma (U-118 MG, ATCC), brain p53 expression glioblastoma (LNZTA3WT4, ATCC), brain glioma (Hs 683, ATCC), glomus kidney glomangioma (glomotel, ATCC), bone eosinophilic granuloma (Hs 454.T, ATCC), lymph node noncaseating granuloma (Hs 697.Ln, ATCC), bone periostitis granuloma (Hs 709.T, ATCC), liver hepatoma (PLC/PRF/5, ATCC), connective tissue histiocytoma (Hs 856.T, ATCC), kidney hypernephroma (SW 156, ATCC), skin keratoacanthoma (Hs 892.T, ATCC), skin malignant acanthocytosis keratoacanthoma (Hs 898.T, ATCC), muscle leiomyosarcoma (TE 149.T, ATCC), uterus leiomyosarcoma (SK-UT-1, ATCC), vulva leiomyosarcoma (SK-LMS-1, ATCC), B lymphoblast acute lymphoblastic leukemia (SUP-B15, ATCC), myeloblast bone marrow acute lymphoblastic leukemia (KG-1, ATCC), T lymphoblast acute lymphoblastic leukemia (MOLT-4, ATCC), monocyte acute monocytic leukemia (THP-1, ATCC), peripheral blood acute myeloid leukemia (AML14.3D10, ATCC), promyeloblast acute promyelocytic leukemia (HL-60, ATCC), T lymphocyte acute T cell leukemia (J.CaM1.6, ATCC), peripheral blood chronic myeloblastic leukemia (Kasumi-4, ATCC), myelomonoblasktic leukemia (GDM-1, ATCC), lymphoblast myelmonocytic leukemia (CESS, ATCC), connective tissue liposarcoma (SW 872, ATCC), lymph node lymphogranulomatosis (Hs 268.T, ATCC), B lymphoblast lymphoma (1A2, ATCC), lymph node lymphoma (Hs 313.T, ATCC), cutaneous T lymphocyte lymphoma (HuT 78, ATCC), B lymphocyte Burkitt's lymphoma (EB-3, ATCC), B cell kidney Burkift's lymphoma (HKB-11, ATCC), lymph node lymphocytic lymphoma (Hs 505.T, ATCC), peritoneal effusion B cell lymphoma (JSC-1, ATCC), upper maxilla Burkift's lymphoma (EB1, ATCC), T lymphocyte cutaneous lymphoma (H9, ATCC), B lymphoblast EBV and KSHV positive lymphoma (BC-1, ATCC), macrophage histiocytic lymphoma (U-937, ATCC), lymph node lymphosarcoma (TE175.T, ATCC), cerebellum brain medulloblastoma (D341 Med, ATCC), skin melanoma (Hs 600.T, ATCC), skin amelanotic melanoma (C32TG, ATCC), connective tissue malignant melanoma (Hs 934.T, ATCC), skin malignant melanoma (A375.S2, ATCC), brain neuroblastoma (CHP-212, ATCC), neuroblast brain neuroblastoma (IMR-32, ATCC), brain neuroglioma (H4, ATCC), bone osteosarcoma (143.98.2, ATCC), connective tissue osteosarcoma (Hs 864.T, ATCC), pharynx papilloma (Hs 840.T, ATCC), B lymphocyte myeloma plasmacytoma (RPMI 8226, ATCC), bone marrow myeloma plasmacytoma (NC1-H929, ATCC), retina retinoblastoma (Y79, ATCC), connective tissue rhabdomyosarcoma (TE 441.T, ATCC), muscle rhabdomyosarcoma (A-673, ATCC), kidney renal rhabdomyosarcoma (Hs 926.T, ATCC), bone sarcoma (SK-ES-1, ATCC), bone giant cell sarcoma (Hs 706.T, ATCC), connective tissue giant cell sarcoma (Hs 127.T, ATCC), vertebral column giant cell sarcoma (Hs 814.T, ATCC), skin pagetoid sarcoma (Hs 925.T, ATCC), lymph node reticulum cell sarcoma (Hs 324.T, ATCC), connective tissue synovial sarcoma (Hs 701.T, ATCC), synovium sarcoma (SW 982, ATCC), uterus sarcoma (MES-SA/MX2, ATCC), bone Ewing's sarcoma (Hs 822.T, ATCC), ovary teratoma (TE 84.T, ATCC), bone sacrococcygeal teratoma (TE 76.T, ATCC), nullipotent stem cell teratocarcinoma (NCCIT, ATCC), cerebellum brain malignant primitive neuroectodermal tumor (PFSK-1, ATCC), oral nonneoplastic tumor (Hs 53.T, ATCC), skin xanthogranuloma (Hs 156.T, ATCC); dog cancer cell lines including connective tissue cancer (CF17.T, ATCC), mammary gland cancer (CF33.MT, ATCC), bone osteosarcoma (D17, ATCC), connective tissue osteosarcoma (CF11.T, ATCC), macrophage histiocytosis (DH82ECOK, ATCC); cat cancer cell lines including bone marrow erythroleukemia (F25, ATCC), connective tissue fibrosarcoma (FC77.T, ATCC), spleen fibrosarcoma (FC81.Sp, ATCC), thymus fibrosarcoma (FC81.Thy, ATCC), lymph node lymphoma (F1B, ATCC) lymphoblast lymphoma (FL74-UCD-1, ATCC), spleen lymphoma (FC16.Sp, ATCC), connective tissue sarcoma (FC100.T, ATCC), spleen sarcoma (FC100.Sp, ATCC), bone marrow reticulum cell sarcoma (FC11.BM, ATCC), thymus osteosarcoma (FC95.Thy, ATCC); mouse cancer cell lines including mammary gland adenocarcinoma (JC, ATCC), pancreas adenocarcinoma (LTPA, ATCC), salivary gland adenocarcinoma (WR21, ATCC), kidney renal adenocarcinoma (RAG, ATCC), lung adenoma (LA-4, ATCC), connective tissue cancer (MM37T, ATCC), mammary gland cancer (MM2SCT, ATCC), colon carcinoma (CT26.WT, ATCC), Lewis lung carcinoma (LL/2, ATCC), lung squamous cell carcinoma (KLN 205, ATCC), bladder fibrosarcoma (MM45T.BI, ATCC), connective tissue fibrosarcoma (MM47T, ATCC), spleen fibrosarcoma (MM45T.Sp, ATCC), liver hepatoma (Hepa 1-6, ATCC), B lymphocyte leukemia (CW13.20-3B3, ATCC), spleen erythroblast leukemia (BB88, ATCC), B lymphocyte lymphoma (WEHI-231, ATCC), monocyte/macrophage lymphoma (P388D, ATCC), spleen lymphoma (RAW 309F.1.1, ATCC), T lymphocyte lymphoma (S1A.TB.4.8.2, ATCC), thymus T lymphocyte lymphoma (R1.1, ATCC), thymus lymphoma (EL4.IL-2, ATCC), mast cell mastocytoma (P815, ATCC), skin melanoma (B16-F10, ATCC), neuroblast brain neuroblastoma (NB41A3, ATCC), B lymphocyte myeloma plasmacytoma (P1.17, ATCC), connective tissue sarcoma (EHS, ATCC), B lymphocyte reticulum cell sarcoma (×16C8.5, ATCC), monocyte/macrophage reticulum cell sarcoma, (J774A.1, ATCC), testis teratocarcinoma (NULLI-SCC1, ATCC), keratinocyte teratoma (XB-2, ATCC); rat cancer cell lines including mammary gland adenocarcinoma (NMU, ATCC), small intestine adenocarcinoma (IA-XsSBR, ATCC), mammary gland cancer (Rn1T, ATCC), prostate cancer (R-3327-AT-1, ATCC), mammary gland carcinoma (DSL-6A/C1, ATCC), pancreas carcinoma (DSL-6A/C1, ATCC), prostate malignant carcinoma (AT3B-1, ATCC), nasal squamous cell carcinoma (FAT 7, ATCC), brain glioma (C6, ATCC), liver hepatoma (H4TG, ATCC), peripheral blood basophil leukemia (RBL-1, ATCC), central nervous system neuroblastoma (B35, ATCC), bone osteosarcoma (UMR-106, ATCC), adrenal gland pheochromocytoma (PC-12, ATCC); Syrian golden hamster skin malignant melanoma (RPMI 1846, ATCC); guinea pig colon colorectal adenocarcinoma (GPC-16, ATCC); chicken hepatocellular liver carcinoma (LMH, ATCC) and bursa lymphoma (DT40, ATCC); bovine cancer cell line including lymph node leukemia (2FLB.Ln, ATCC), B lymphocyte lymphosarcoma (BL3.1, ATCC), bone marrow lymphosarcoma (LB9.Bm, ATCC), spleen lymphosarcoma (LB10.Sp, ATCC), thymus lymphosarcoma (LB9.Thy, ATCC) and any other naturally occurring cancers from any species. Cancer vaccines made from these cancer tumor or cell lines may be used to produce vaccine specific immune components to kill corresponding cancer cells or tumors in in vitro, in vivo and in situ settings or clinical trials for human and animals.

Due to genomic differences, cancer vaccines made from cancer cells of one species are useful only for the same species to fight against cancer cells. For example, human cancer vaccines made from human cancer cell lines or tumor lines must be used for human cancer prevention or treatment of cancer. Human cancer vaccines should not be used for any animal vaccinations, and vice versa. For an immune competent animal, human cancer vaccine or human cancer cells are both foreign and can induce immune responses. However, these immune responses are against human cancer cells, not against any animal cancer cells. Nevertheless, humanized antibodies against human cancer vaccines made from various systems including animals may be useful for human cancer patients' immunotherapy.

Another example is that cat cancer vaccines made from cat cancer cell lines will not prevent dogs' cancer, vice versa. Although a cat's cancer vaccine may induce immune responses in dogs, any cat's cancer never naturally occur in dogs. Thus, dogs vaccinated with a cat cancer vaccine may not prevent any dog cancer. Furthermore, human's breast cancer vaccine may not be used to prevent human's prostate cancer if the mutation profile in breast cancer vaccine antigens does not cover any prostate cancer cell associated antigens.

Because a cancer vaccine is harmless, multiple cancer vaccines' vaccinations may induce multiple immune responses against multiple cancers. Multiple sets of immune components isolated from individuals with multiple cancer vaccines' vaccination may be isolated for more effective immunotherapy on cancer. Immune components include, but are not limited to, polyclonal antibodies and activated lymphocytes such as B-cells, T-cells, macrophages, monocytes and natural killer cells. The cancer vaccine specific immune components may be obtained from the blood of vaccinated individuals. These immune components may be used to kill cancer cells for cancer patients who are compatible with blood donor's blood types but have a suppressed immune system that does not sufficiently respond to the cancer vaccine.

1. A harmless cancer vaccine is derived from cancer cells with extracellular proteins being digested by a proteinase during the vaccine preparation and used to induce an individual's immune responses against cancer cells.

2. Cancer vaccine specific immune components are responsible for cancer vaccine induced immune responses against cancer cells.

3. Claim 1 wherein the proteinase is Tumorase™.

4. Claim 1 wherein a proteinase is selected from a list consisting of:
carboxypeptidase B, elastase, plasmin, endoproteinase Glu-C, endoproteinase Asp-N, endoproteinase Lys-C, endoproteinase Arg-C, chymotrypsin, or carboxypeptidase Y, caspases, proteinase K, subtilisin BL, M-protease, thermitase, subtilisin Carlsberg, subtilisin Novo BPN′, subtilisin BPN′, selenosubtilisin, tonin, blood coagulation factor XA, rat mast cell protease II, kallikrein A, pronase, trypsin, anhydro-trypsin, beta-trypsin, alpha-chymotrypsin, gamma-chymotrypsin, elastase, tosyl-elastase, human neutrophil elastase, human leukocyte elastase, alpha-thrombin, gamma-thrombin, epsilon-thrombin, glutamic acid specific protease, achromobacter protease I, alpha-lytic protease, proteinase A, proteinase B, actinidin, cathepsin B, papaya protease omega, papain, interleukin 1-beta converting enzyme, myeloblastosis associated viral protease, rous sarcoma virus protease, simian immunodeficiency virus protease, HIV-1 protease, HIV-2 protease, cathepsin D, chymosin B, endothiapepsin, penicillopepsin, pepsin, pepsin 3A, renin, rhizopuspepsin, neutral protease, thermolysin, astacin, astacin (zinc replaced by Cu2+), astacin (zinc replaced by cobalt2+), astacin (zinc replaced by mercury2+), astacin (zinc removed), astacin (zinc replaced by nickel2+), serralysin (bound to zinc), collagenase, fibroblast collagenase and neutrophil collagenase.

5. Claim 1 wherein cancer cells become harmless by a proteinase digestion and a further process or processes selected from the group consisting of:
proteinase digestion, formalin, phenol, heat, freeze-thaw-freeze, y-ray, x-ray, microwave and UV.

6. Claim 1 wherein cancer cells are from tissue-cultured animal cancer cell lines.

7. Claim 1 wherein cancer cells are from tissue-cultured human cancer cell lines.

8. Claim 1 wherein cancer cells are from an animal cancer patient.

9. Claim 1 wherein cancer cells are from a human cancer patient.

10. Claim 1 wherein the harmless cancer vaccine induced immune responses are for cancer prevention.

11. Claim 1 wherein an individual is selected from the group consisting of:
human, mouse, dog, cat, hamster, horse, rabbit, rat, chicken, cow, tiger, panda, pig, sheep or monkey.

12. Claim 2 wherein cancer vaccine specific immune components are polyclonal antibodies, B-cells, T-cells, natural killer cells, monocytes, dendritic cells and macrophages.

13. Claim 2 wherein cancer vaccine specific immune components are used to help a compatible individual's immune system to kill cancer cells.

14. Claim 2 wherein cancer cells are selected from the group consisting of:
Cell culture in vitro, tissue culture in vitro, organ culture in vitro, cells grown in nude mouse, tissue grown in nude mouse or organ grown in nude mouse.

15. Claim 2 wherein cancer cells are not forming tumors.

16. Claim 2 wherein cancer cells are forming malignant tumors.

17. Claim 2 wherein cancer cells are forming micrometastasis.

18. Claim 2 wherein cancer cells are forming malignant solid tumors.

19. Claim 2 wherein cancer cells are forming tumors deep inside the body.

20. Claim 2 wherein cancer cells are in the body of an animal.

21. Claim 2 wherein cancer cells are in the body of human.
